(19) United States
(12) Patent Application Publication
(10) Pub. No.: US 2022/0313102 A1
(43) Pub. Date: Oct. 6, 2022

(54) PARACENTESIS ASSISTANCE SYSTEM, PARACENTESIS ASSISTANCE METHOD, AND PROGRAM

(71) Applicant: OSAKA UNIVERSITY, Suita-shi, Osaka (JP)

(72) Inventors: Yo KOBAYASHI, Suita-shi, Osaka (JP); Chiyo OOTAKI, Suita-shi, Osaka (JP)

(21) Appl. No.: 17/639,483

(22) PCT Filed: Sep. 1, 2020
(86) PCT No.: PCT/JP2020/032994

§ 371 Date: Mar. 1, 2022

(30) Foreign Application Priority Data
Sep. 2, 2019 (JP) 2019-159308

(51) Int. Cl. A61B 5/053; A61B 5/00

(52) U.S. Cl. CPC A61B 5/053; A61B 5/4887

(57) ABSTRACT

Provided is a paracentesis assistance system that identifies the type of biological tissue. The paracentesis assistance system (10) comprises a measurement device that applies high-frequency waves to at least two electrodes (31 and 32) of an electrode needle (3) inserted into a biological tissue (9), and repeatedly measures the electrical impedance of the biological tissue (9) where the electrode (31) is located, the electrodes being arranged at the tip of the electrode needle in a longitudinal direction; and an identification device (2) that identifies the type of biological tissue (9) based on the temporal change in the repeatedly measured electrical impedance.

### TECHNICAL FIELD

The present invention relates to a system that assists the puncture of a biological tissue with an electrode needle.

### BACKGROUND ART

A nerve block is a useful method that is frequently applied to both clinical and practice applications for intraoperative and postoperative analgesia. In recently widely used methods, while performing a nerve block using a puncture needle and an anesthetic, the position of the needle tip is estimated under the guidance of ultrasound (US) images using an ultrasound diagnostic device.

Further, as a technique for reliably puncturing a target biological tissue with a needle, for example, PTL 1 discloses a technique of detecting the puncture of the cardiac tissue with an injection needle based on the change in the measured impedance values. PTL 2 discloses a technique of detecting the progress of the distal tip of a needle from the measured bioimpedance.

### CITATION LIST

### Patent Literature

PTL 1: JP2004-290583A
PTL 2: WO2009/083651

### SUMMARY OF INVENTION

### Technical Problem

In order to perform a nerve block accurately and safely, it is required that the target nerve is not damaged by the needle, and that the local anesthetic is injected accurately around the target nerve. To ensure this, the operator is required to place the needle tip as close as possible to the target nerve while maintaining an appropriate distance.

However, it is not easy to distinguish the target nerve from the surrounding tissues. For example, in the field of anesthesiology, it is required that nerve tissue is distinguished from muscle tissue or adipose tissue, and that central nerve is distinguished from peripheral nerve. Accurate identification of the types of these biological tissues is directly linked to the success or failure of nerve blocks; however, it is not easy to accurately identify the types of these biological tissues only from ultrasound images. In order to perform the procedure of nerve block accurately and safely only under the guidance of ultrasound images, the operators are still expected to have a knowledge of ultrasound anatomy and a good understanding of ultrasound images. In order to perform nerve blocks more accurately and safely, the operators are expected to be able to accurately identify the type of biological tissue. Neither the technique of PTL 1 nor the technique of PTL 2 can be used to identify the type of biological tissue.

The present invention provides a paracentesis assistance system, method, and program that identify the type of biological tissue.

### Solution to Problem

In order to achieve the above object, the present invention includes, for example, the following embodiments.

(Item 1)

A paracentesis assistance system comprising:

a measurement device that applies high-frequency waves to at least two electrodes of an electrode needle inserted into a biological tissue, and repeatedly measures the electrical impedance of the biological tissue where the electrodes are located, the electrodes being arranged at the tip of the electrode needle in a longitudinal direction; and

an identification device that identifies the type of biological tissue based on the temporal change in the repeatedly measured electrical impedance.

(Item 2)

The paracentesis assistance system according to Item 1, wherein the identification device calculates a time average of the electrical impedance within a predetermined period of time, and compares the calculated time average with a database that associates the type of biological tissue with a reference value of the time average, thereby identifying the type of biological tissue.

(Item 3)

The paracentesis assistance system according to Item 2, further comprising a notification unit that gives notification depending on an identification result by the identification device.

(Item 4)

The paracentesis assistance system according to Item 3, wherein the notification unit gives notification when the calculated time average is outside a predetermined value range including the reference value.

(Item 5)

The paracentesis assistance system according to any one of Items 1 to 4, wherein the identification device calculates the amount of change in the electrical impedance within a predetermined period of time, and identifies that the type of biological tissue changes when the calculated amount of change is within a predetermined value range.

(Item 6)

The paracentesis assistance system according to Item 5, wherein the identification device calculates the amount of change in the electrical impedance within a predetermined period of time based on |EI₂−EI₁|/EI₁ or |EI₂−EI₁|/EI₂, wherein EI₁ is the measured value of the electrical impedance of a first tissue, and EI₂ is the measured value of the electrical impedance of a second tissue, when the type of biological tissue located at the tip of the electrode needle changes from the first tissue to the second tissue.

(Item 7)

The paracentesis assistance system according to Item 5 or 6, wherein the identification device identifies whether the electrodes are located in a nerve tissue.

(Item 8)

The paracentesis assistance system according to Item 5 or 6, wherein the identification device identifies whether the electrodes are located in a biological tissue between muscles.

(Item 9)

The paracentesis assistance system according to Item 7, further comprising an electrical stimulus generator that applies an electrical pulse to the electrodes to stimulate the biological tissue.

(Item 10)

The paracentesis assistance system according to Item 7, wherein the electrode needle is hollow, and an anesthetic is injected through the electrode needle into the biological tissue where the electrodes are located.

(Item 11)

A paracentesis assistance method comprising:

applying high-frequency waves to at least two electrodes of an electrode needle inserted into a biological tissue, and repeatedly measuring the electrical impedance of the biological tissue where the electrodes are located, the electrodes being arranged at the tip of the electrode needle in a longitudinal direction; and

identifying the type of biological tissue based on the temporal change in the repeatedly measured electrical impedance.

(Item 12)

A program for causing a computer to realize:

a function of applying high-frequency waves to at least two electrodes of an electrode needle inserted into a biological tissue, and repeatedly measuring the electrical impedance of the biological tissue where the electrodes are located, the electrodes being arranged at the tip of the electrode needle in a longitudinal direction; and

a function of identifying the type of biological tissue based on the temporal change in the repeatedly measured electrical impedance.

### Advantageous Effects of Invention

The present invention can provide a paracentesis assistance system, method, and program that identify the type of biological tissue.

### BRIEF DESCRIPTION OF DRAWINGS

FIG. 1 shows a schematic structure of a paracentesis assistance system according to one embodiment of the present invention.

FIG. 2 shows a schematic structure of the tip of an electrode needle.

FIG. 3 is a block diagram for explaining the function of an identification device according to one embodiment of the present invention.

FIG. 4 is a flowchart for explaining the procedure of a paracentesis assistance method using the paracentesis assistance system according to one embodiment of the present invention.

FIG. 5 is a flowchart for explaining the procedure of a paracentesis assistance method using the paracentesis assistance system according to one embodiment of the present invention.

FIG. 6 shows examples of screen display in the paracentesis assistance system according to one embodiment of the present invention.

FIG. 7 shows an example of ultrasound images in Example 1.

FIG. 8 is a graph showing the average of electrical impedance values inside and outside the sciatic nerve in Example 1.

FIG. 9 is a graph showing the time change in electrical impedance values during puncture in Example 1.

FIG. 10 shows an example of ultrasound images in Example 2.

FIG. 11 is a graph showing the average of electrical impedance values in the internal oblique muscle and a tissue surface in Example 2.

FIG. 12 is a graph showing the time change in electrical impedance values during puncture in Example 2.

FIG. 13 shows an example of ultrasound images in Example 3.

FIG. 14 is a graph showing the change in the measured value of electrical impedance when the position of the tip of the electrode needle changes from the muscle to the sciatic nerve sheath in Example 3.

### DESCRIPTION OF EMBODIMENTS

An embodiment of the present invention is described in detail below with reference to the attached drawings. In the following description and drawings, the same reference signs indicate the same or similar components. Therefore, duplicate descriptions of the same or similar components are omitted.

### SUMMARY OF INVENTION

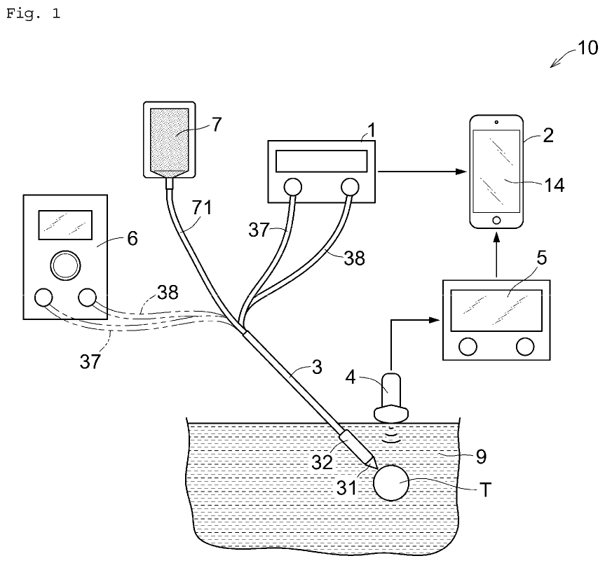

FIG. 1 shows a schematic structure of a paracentesis assistance system according to one embodiment of the present invention.

The use mode of the paracentesis assistance system 10 according to one embodiment is described with reference to FIG. 1. The paracentesis assistance system 10 according to one embodiment comprises a measurement device 1 and an identification device 2, and identifies the type of biological tissue 9 located at the tip of an electrode needle 3 punctured into the biological tissue 9. The type of biological tissue 9 is identified based on the temporal change in the electrical impedance of the biological tissue 9.

The electrode needle 3 is gradually punctured from its tip into the biological tissue 9 by the technique of the operator. During the puncture technique by the operator, the paracentesis assistance system 10 repeatedly measures the electrical impedance by the measurement device 1, and repeatedly identifies the type of biological tissue 9 by the identification device 2. Thereafter, the puncture technique is advanced, and when the paracentesis assistance system 10 identifies the type of biological tissue 9 located at the tip of the electrode needle 3 as a target tissue T, the paracentesis assistance system 10 notifies the operator accordingly. The operator determines that the tip of the electrode needle 3 is located in the vicinity of the target tissue T, and completes the puncture technique.

In the present embodiment, the puncture technique by the operator is performed under the guidance of ultrasound images captured by using an ultrasound probe 4 and an ultrasound diagnostic device 5, and the captured ultrasound images are displayed in a display unit 14 of the identification device 2. The ultrasound images may be displayed in a display unit of the ultrasound diagnostic device 5.

In the present embodiment, the biological tissue 9 is a human biological tissue, and the target tissue T is a nerve tissue (e.g., sciatic nerve). In the present embodiment, after completion of the puncture technique, the operator can use an electrical stimulus generator 6 to stimulate the biological tissue 9, thereby reconfirming whether the biological tissue 9 located at the tip of the electrode needle 3 is truly the nerve tissue (sciatic nerve). Thereafter, the operator can inject an anesthetic 7 through the hollow electrode needle 3 around the biological tissue 9 (i.e., target tissue T) that has been identified as the nerve tissue.

In the paracentesis assistance system 10 according to one embodiment, the measurement device 1 and the identification device 2 are configured as separate devices; however, the measurement device 1 and the identification device 2 may be integrated to form a single paracentesis assistance device. Further, such a paracentesis assistance device may be suitably integrated with the ultrasound diagnostic device 5, the electrical stimulus generator 6, and the like. In the description of the present specification and claims, the term “system” means not only a system configured using a plurality of individually independent devices, but also a device configured by integrating a plurality of devices.

### Structure of Paracentesis Assistance System

The structure of the paracentesis assistance system 10 according to one embodiment is described with reference to FIG. 1.

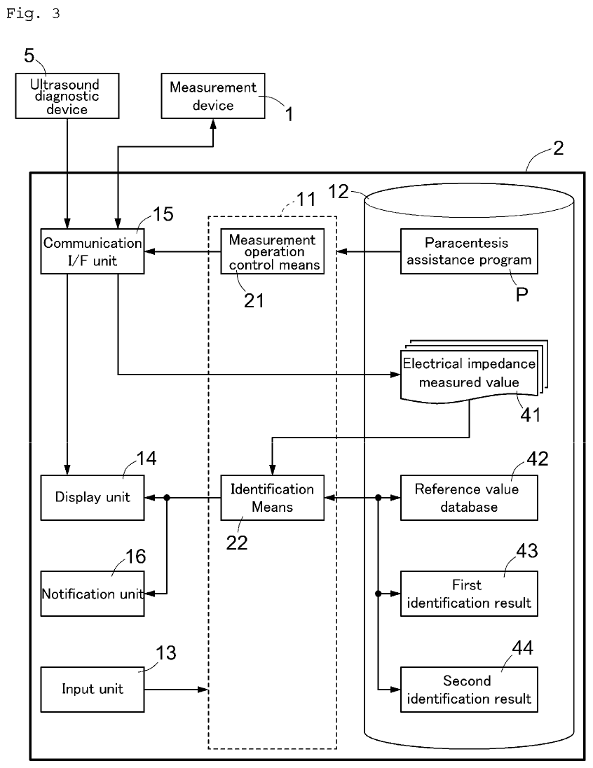

The paracentesis assistance system 10 according to one embodiment comprises a measurement device 1 and an identification device 2. The measurement device 1 applies high-frequency waves to electrodes 31 and 32 of an electrode needle 3, and repeatedly measures the electrical impedance of a biological tissue 9 where the electrode 31 is located. The measurement device 1 can be a known impedance analyzer. The identification device 2 identifies the type of biological tissue 9 based on the temporal change in the repeatedly measured electrical impedance. The identification device 2 is described later with reference to FIG. 3.

The electrode needle 3, an ultrasound probe 4, an ultrasound diagnostic device 5, and an electrical stimulus generator 6 are used together with the paracentesis assistance system 10. The electrical stimulus generator 6 is an optional structure.

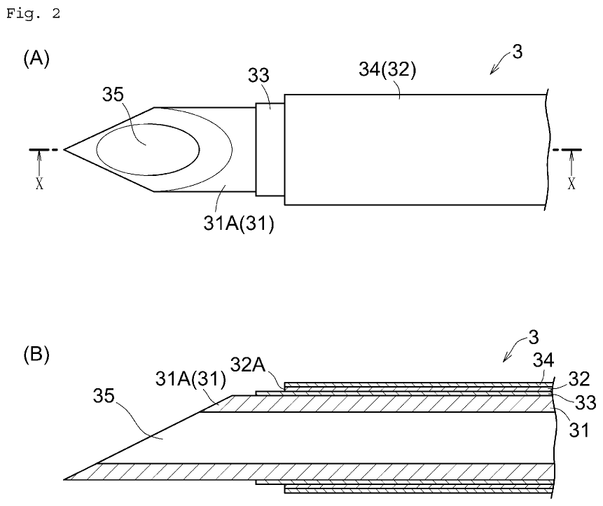

In the electrode needle 3 at its tip to be punctured into the biological tissue 9, at least two electrodes 31 and 32 are arranged in a longitudinal direction. When the electrodes 31 and 32 are connected to the measurement device 1 through connection codes 37 and 38, they function as electrodes for measuring the electrical impedance of the biological tissue 9. When the electrodes 31 and 32 are connected to the electrical stimulus generator 6 through the connection codes 37 and 38, they function as electrodes for applying an electrical pulse for electrically stimulating the biological tissue 9. In the present embodiment, the electrode needle 3 is hollow, and an anesthetic 7 is injected through the electrode needle 3 into the biological tissue 9 where the electrode 31 is located. The electrode needle 3 is described later with reference to FIG. 2.

The ultrasound probe 4 and the ultrasound diagnostic device 5 capture ultrasound images around the biological tissue 9 to be subjected to the puncture technique. The ultrasound probe 4 and the ultrasound diagnostic device 5 can be known ultrasound diagnostic devices. The electrical stimulus generator 6 applies an electrical pulse to the electrodes 31 and 32 to stimulate the biological tissue 9. The electrical stimulus generator 6 can be a known neuromuscular electrical stimulator.

FIG. 2 shows a schematic structure of the tip of the electrode needle. (A) is a plan view of the tip of the electrode needle 3, and (B) is a cross-sectional view taken along line X-X in (A).

The electrode needle 3 according to one embodiment comprises a cylindrical internal electrode needle 31 with a pointed end part 31A, and a cylindrical external electrode needle 32 exposing the pointed end part 31A and covering the outer surface of the internal electrode needle 31. The electrode needle 3 is punctured into the biological tissue 9 along the longitudinal direction. An insulation layer 33 is formed between the internal electrode needle 31 and the external electrode needle 32, and the external electrode needle 32 is electrically insulated from the internal electrode needle 31. The insulation layer 33 is formed to expose the pointed end part 31A. An insulation layer 34 is formed on the outer surface of the external electrode needle 32. The pointed end part 31A of the internal electrode needle 31 exposed from the insulation layer 33, and an end face 32A of the external electrode needle 32 exposed from the insulation layer 33 and insulation layer 34 are arranged in the longitudinal direction (puncture direction) of the electrode needle 3.

The internal electrode needle 31 and the external electrode needle 32 are connected to the measurement device 1 through the connection codes 37 and 38. The internal electrode needle 31 and the external electrode needle 32 can also be connected to the electrical stimulus generator 6 through the connection codes 37 and 38.

The internal electrode needle 31 and the external electrode needle 32 are made of conductive metal. The insulation layers 33 and 34 are formed, for example, by a coating of a fluoropolymer resin having an insulating property. In the present embodiment, the internal electrode needle 31 is hollow, and a drug solution (e.g., anesthetic 7) is injected around the target tissue T through a hollow space 35 of the internal electrode needle 31.

### Structure of Identification Device

FIG. 3 is a block diagram for explaining the function of an identification device according to one embodiment of the present invention.

The identification device 2 according to one embodiment comprises a data processing means 11, an auxiliary storage device 12, an input unit 13, a display unit 14, a communication interface unit (communication I/F unit) 15, and a notification unit 16. The identification device 2 can be configured using, for example, a tablet terminal or a smartphone (hereinafter referred to as “tablet terminal or the like”).

In the present embodiment, the identification device 2 comprises the auxiliary storage device 12, the input unit 13, the display unit 14, the communication I/F unit 15, and the notification unit 16 as hardware configurations. Although it is not shown, the identification device 2 further comprises a processor, such as a CPU, for data processing, and a memory used by the processor for the work area for data processing, as hardware configurations.

The auxiliary storage device 12 is a non-volatile storage device that stores an operating system (OS), various control programs, data generated by the programs, and the like, and is configured from, for example, a flash memory, eMMC (embedded Multi Media Card), SSD (Solid State Drive), etc. In the present embodiment, the auxiliary storage device 12 stores a measured value 41 of electrical impedance, a reference value database 42, and a paracentesis assistance program P.

The measured value 41 of electrical impedance is the measured value of the electrical impedance of the biological tissue 9 where the electrode 31 is located, and is measured by the measurement device 1.

The reference value database 42 is a database that associates the type of biological tissue 9 with reference values of the time average of electrical impedance within a predetermined period of time.

The paracentesis assistance program P is a computer program for realizing means 21 and 22 in the data processing means 11, described later, which is a functional block by software. The paracentesis assistance program P can be installed in the identification device 2 via a network, such as the Internet, connected through the communication I/F unit 15. Alternatively, the paracentesis assistance program P may be installed in the identification device 2 by causing the identification device 2 to read a computer-readable non-transitory tangible recording medium (e.g., a memory card) that records the paracentesis assistance program P. The paracentesis assistance program P can be, for example, an application of a tablet terminal or the like.

The input unit 13 can be configured with, for example, a mouse and a keyboard, and the display unit 14 can be configured with, for example, a liquid crystal display or an organic EL display. In the present embodiment, the input unit 13 and the display unit 14 are integrated as a touch panel.

The communication I/F unit 15 transmits and receives data to and from external devices, such as the measurement device 1 and the ultrasound diagnostic device 5, through a wired or wireless network. The communication I/F unit 15 may have various wireless connections or wired connections, such as Bluetooth (registered trademark), Wi-Fi (registered trademark), and ZigBee (registered trademark).

The notification unit 16 gives notification according to the identification result by the identification device 2 based on an operation instruction from the identification means 22. For example, the notification unit 16 can generate a caution signal when the type of biological tissue 9 changes, and can generate a danger signal when the biological tissue 9 is not present in the database 42. The caution signal is, for example, a low-frequency beep, and the danger signal is, for example, a high-frequency beep. The notification unit 16 can give notification when the time average of electrical impedance within a predetermined period of time is outside a predetermined value range, including the reference values of the time average. In the present embodiment, the notification unit 16 is a buzzer or a speaker, and gives notification to the operator by generation of sound. The notification unit 16 is not limited to a buzzer or a speaker, and may be, for example, a vibrator or an indicator composed of LED light etc. The notification unit 16 may be configured to be able to notify the operator of the identification result by the identification means 22.

In the present embodiment, the identification device 2 comprises the data processing means 11 as a software configuration. The data processing means 11 is a functional block realized by executing the paracentesis assistance program P by the processor.

A measurement operation control means 21 controls the measurement operation performed by the measurement device 1. By the control of the measurement operation control means 21, the measurement device 1 applies high-frequency waves to the electrodes 31 and 32 of the electrode needle 3, repeatedly measures the electrical impedance of the biological tissue 9 where the electrode 31 is located, and stores the measured value 41 of electrical impedance in the auxiliary storage device 12. The measurement device 1 repeatedly performs the measurement operation with a predetermined measurement cycle (i.e., at predetermined time intervals).

The identification means 22 calculates the time average of the electrical impedance 41 within a predetermined period of time, and compares the calculated time average with the database 42, which associates the type of biological tissue 9 with the reference values of the time average, thereby identifying the type of biological tissue 9.

Further, the identification means 22 calculates the amount of change in the electrical impedance 41 within a predetermined period of time. When the calculated amount of change is within a predetermined value range, the identification means 22 can identify that the type of biological tissue 9 changes. In the present embodiment, the identification means 22 identifies whether the electrode 31 is located in a nerve tissue (e.g., sciatic nerve).

In the present embodiment, the predetermined period of time is determined based on the measurement cycle. That is, in the present embodiment, the identification means 22 calculates the time average of the electrical impedance 41 from the average of the measured value of the electrical impedance 41 in the current measurement cycle and the measured value of the electrical impedance 41 in the immediately preceding measurement cycle, and calculates the amount of change in the electrical impedance 41 from the difference between the measured value of the electrical impedance 41 in the current measurement cycle and the measured value of the electrical impedance 41 in the immediately preceding measurement cycle.

### Processing Procedure

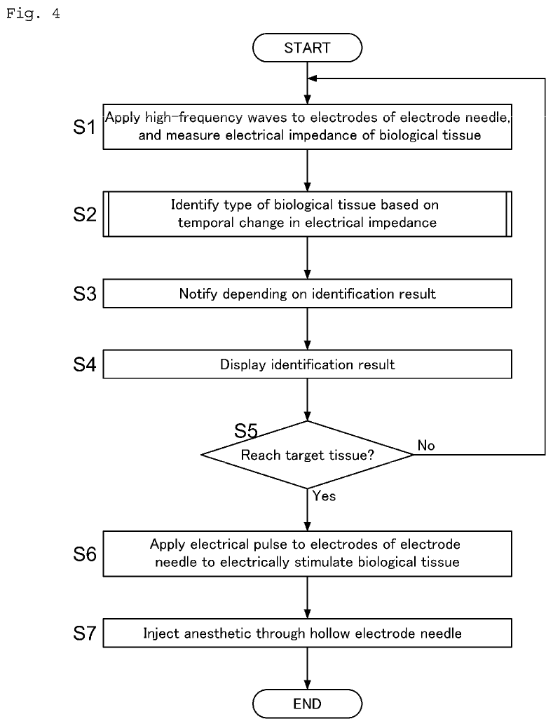

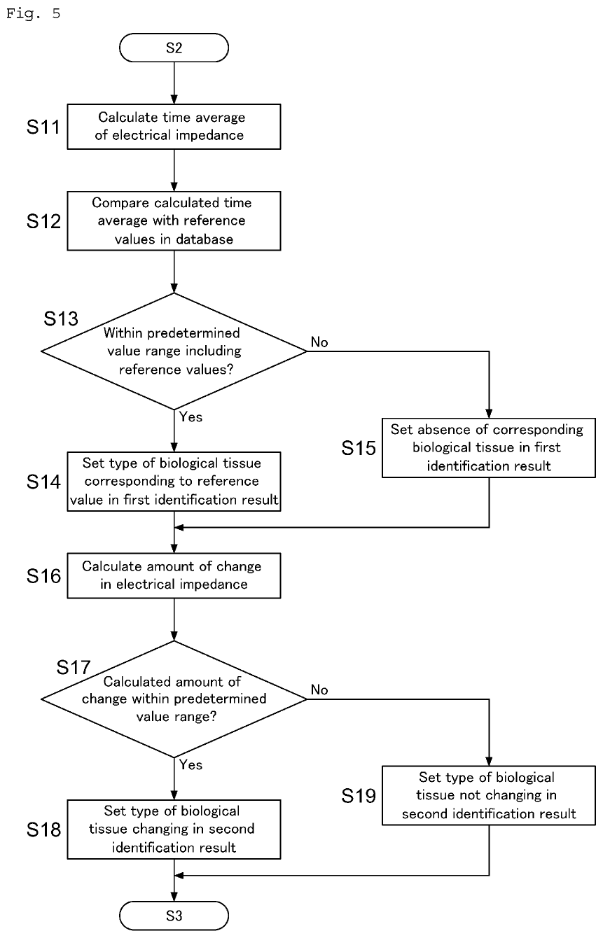

FIGS. 4 and 5 show flowcharts for explaining the procedure of a paracentesis assistance method using the paracentesis assistance system according to one embodiment of the present invention.

First, by the technique of the operator, the electrode needle 3 is gradually punctured from its tip, specifically the pointed end part 31A of the internal electrode needle 31, towards the target tissue T in the biological tissue 9. During the puncture technique by the operator, the paracentesis assistance system 10 repeatedly performs the processing of the following steps S1 to S5.

In step S1 (measurement step), high-frequency waves are applied to the electrodes 31 and 32 of the electrode needle 3, and the electrical impedance of the biological tissue 9 where the electrode 31 is located is repeatedly measured.

In step S2 (identification step), the type of biological tissue 9 is identified based on the temporal change in the repeatedly measured electrical impedance.

The identification step as step S2 is described in detail with reference to FIG. 5. In step S2, the processing of steps S11 to S19 is performed.

In steps S11 to S15, the type of biological tissue 9 is identified. The identification result is stored as a first identification result 43 in the auxiliary storage device 12, for example.

In step S11, the time average of the electrical impedance 41 within a predetermined period of time is calculated. The time average of the electrical impedance 41 is calculated from the average of the measured value of the electrical impedance 41 in the current measurement cycle and the measured value of the electrical impedance 41 in the immediately preceding measurement cycle.

In step S12, the calculated time average is compared with the reference values in the reference value database 42. Table 1 shows examples of the reference value database 42 related to the time average of the electrical impedance 41. The values shown in Tables 1 to 4 were measured and determined using rabbits as targets in the Examples described later. In the present embodiment, the following description is provided on the assumption that the same numerical values as those in the Examples can also be applied to humans, for convenience of explanation.

TABLE 1

| Electrical impedance value | |
|---|---|
| time average [kΩ] | Biological tissue name |
| 4.51 ± 0.71 | Outside sciatic nerve (muscle or |
| | adipose tissue) |
| 2.68 ± 0.67 | Inside sciatic nerve (sciatic nerve |
| | sheath or sciatic nerve itself) |

It is determined in step S13 whether the calculated time average is within a predetermined value range, including the reference values. When the calculated time average is within the range (Yes in step S13), the type of biological tissue 9 corresponding to the reference value is set in the first identification result 43 in step S14, and the processing proceeds to step S16. When the calculated time average is outside the range (No in step S13), the absence of the biological tissue 9 corresponding to any of the reference values is set in the first identification result 43 in step S15, and the processing proceeds to step S16.

For example, with reference to Table 1, it is determined whether the calculated time average of the electrical impedance 41 is within the range of 4.51±0.71 kΩ, and whether the calculated time average of the electrical impedance 41 is within the range of 2.68±0.67 kΩ. When the calculated time average of the electrical impedance 41 is within the range of 4.51±0.71 kΩ, the type of biological tissue 9 being a biological tissue outside the sciatic nerve (e.g., muscle or adipose tissue) is set in the first identification result 43. When the calculated time average of the electrical impedance 41 is within the range of 2.68±0.67 kΩ, the type of biological tissue 9 being a biological tissue inside the sciatic nerve (e.g., sciatic nerve sheath or sciatic nerve itself) is set in the first identification result 43. When the calculated time average of the electrical impedance 41 does not fall under either of the time average ranges shown as reference values in Table 1, the absence of the biological tissue 9 corresponding to any of the reference values is set in the first identification result 43.

In steps S16 to S19, it is identified whether the type of biological tissue 9 located at the tip of the electrode needle 3 changes during the puncture technique. The identification result is stored as a second identification result 44 in the auxiliary storage device 12, for example.

In step S16, the amount of change in the electrical impedance 41 within a predetermined period of time is calculated. The amount of change in the electrical impedance 41 is calculated from the difference between the measured value of the electrical impedance 41 in the current measurement cycle and the measured value of the electrical impedance 41 in the immediately preceding measurement cycle.

In step S17, it is determined whether the calculated amount of change is within a range of predetermined values (reference values). For comparison with the calculated amount of change, Table 2 shows an example of the reference values of the amount of change in the electrical impedance 41. In the present embodiment, the reference value of the amount of change in the electrical impedance 41 shown in Table 2 is stored in the reference value database 42, and is referred to for comparison as appropriate.

TABLE 2

| Electrical impedance value | |
|---|---|
| change amount [kΩ] | Event name |
| 1.83 ± 0.74 | Reach sciatic nerve |

When the calculated amount of change is within the range including the reference value of Table 2 (Yes in step S18), the type of biological tissue 9 changing is set in the second identification result 44 in step S19, and the processing proceeds to step S3. When the calculated amount of change is outside the range (No in step S18), the type of biological tissue 9 not changing is set in the second identification result 44 in step S20, and the processing proceeds to step S3.

For example, when the calculated amount of change in the electrical impedance 41 is within the range of 1.83±0.74 kΩ with reference to Table 2, the type of biological tissue 9 changing from the biological tissue outside the sciatic nerve to the biological tissue inside the sciatic nerve, and the tip of the electrode needle 3 reaching the target tissue T are set in the second identification result 44. When the calculated amount of change in the electrical impedance 41 is outside the range of 1.83±0.74 kΩ, the type of biological tissue 9 not changing is set in the second identification result 44.

With reference to FIG. 4 again, notification is given according to the identification result in step S3 (notification step).

For example, when the identification means 22 identifies that there is no biological tissue 9 corresponding to any of the reference values with reference to the first identification result 43, the notification unit 16 generates, for example, a high-frequency beep as a danger signal. Further, for example, when the identification means 22 identifies that the type of biological tissue 9 changes with reference to the second identification result 44, the notification unit 16 generates, for example, a low-frequency beep as a caution signal.

In step S4 (display step), the identification result is displayed.

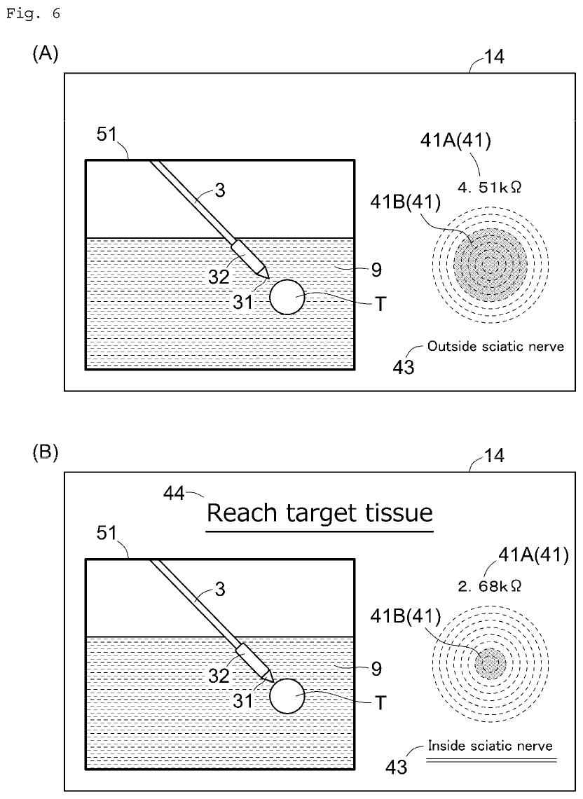

FIG. 6 shows examples of screen display in the paracentesis assistance system according to one embodiment of the present invention. (A) is screen display when the tip of the electrode needle 3 does not reach the target tissue T, and (B) is screen display when the tip of the electrode needle 3 reaches the target tissue T.

As shown in FIG. 6 (A), the identified type of biological tissue 9 is displayed in the display unit 14 as the first identification result 43. In the present embodiment, as shown in FIG. 6, the identification results 43 and 44 are displayed in the display unit 14 together with an ultrasound image 51 obtained from the ultrasound diagnostic device 5. Further, in the present embodiment, the measured value 41 of electrical impedance is displayed in real time and continuously in the display unit 14 using a numerical value 41A and a graphic 41B that represents the size of the value.

Then, the puncture technique is advanced, and, for example, when the tip of the electrode needle 3 reaches the target tissue T, as shown in FIG. 6 (B), the display unit 14 displays the second identification result 44 to indicate that the type of biological tissue 9 changes, and that the tip of the electrode needle 3 reaches the target tissue T.

In step S5, the puncture technique is advanced, and the paracentesis assistance system 10 repeats the processing from step S1 until the tip of the electrode needle 3 reaches the target tissue T, namely until the type of biological tissue 9 located at the tip of the electrode needle 3 is identified as the target tissue T (nerve tissue) (Yes in step S5).

As a result of the processing of steps S1 to S5, the paracentesis assistance system 10 identifies the type of biological tissue 9 located at the tip of the electrode needle 3 as the target tissue T, after which the operator can perform the processing of the following steps S6 to S7 as optional steps.

In step S6 (electrical stimulation step), the electrical stimulus generator 6 is used to apply an electrical pulse to the electrodes 31 and 32 of the electrode needle 3 to stimulate the biological tissue 9. The operator connects the connection codes 37 and 38, which are connected to the measurement device 1, to the electrical stimulus generator 6, thereby electrically stimulating the biological tissue 9. As a result, the operator can reconfirm whether the biological tissue 9 located at the tip of the electrode needle 3 and identified as the target tissue T is truly the nerve tissue.

In step S7 (anesthetic injection step), an anesthetic is injected through the hollow electrode needle 3. In the present embodiment, the hollow space 35 of the internal electrode needle 31 provided in the electrode needle 3, and a syringe filled with a drug solution of the anesthetic 7 are connected through, for example, a drug solution tube 71. The drug solution of the anesthetic 7 is injected around the biological tissue 9 (target tissue T) through the hollow space 35 of the internal electrode needle 31, for example, by the operation of the syringe by the operator. As a result, a nerve block is applied to the biological tissue 9 that is identified as the nerve tissue.

### Effects

With the paracentesis assistance system, method, and program according to one embodiment, the type of biological tissue can be identified. As a result, the operator can easily identify the target biological tissue from other biological tissues during the puncture technique, and can also easily bring the tip of the electrode needle close to the target biological tissue. This makes it possible to perform nerve blocks more accurately and more safely.

### Other Embodiments

Paracentesis assistance systems according to other embodiments described below are similar to the paracentesis assistance system according to one embodiment described above, unless otherwise specified. Accordingly, duplicate descriptions are omitted.

In the above embodiment, the identification means 22 identifies whether the electrode 31 is located in the nerve tissue with reference to the reference values shown in Tables 1 and 2; however, in another embodiment, the identification means 22 identifies whether the electrode 31 is located in a biological tissue between muscles with reference to the reference values shown in Tables 3 and 4. In still another embodiment, the identification means 22 can identify whether the electrode 31 is located in a nerve tissue, and whether the electrode 31 is located in a biological tissue between muscles, with reference to Tables 1 to 4.

Table 3 shows examples of the reference value database 42 that is previously determined in relation to a muscle and a tissue surface between muscles.

TABLE 3

| Electrical impedance value | |
|---|---|
| time average [kΩ] | Biological tissue name |
| 5.41 ± 0.66 | Muscle (internal oblique muscle) |
| 8.79 ± 0.94 | Between muscles (tissue surface |
| | between internal oblique muscle and |
| | transverse abdominal muscle) |

Similarly with steps S11 to S15 according to one embodiment described above, the identification means 22 calculates the time average of the electrical impedance 41 within a predetermined period of time, and identifies the type of biological tissue 9 based on the reference values of the time average of the electrical impedance 41 shown in Table 3. The identification result is stored as a first identification result 43 in the auxiliary storage device 12, for example.

Table 4 shows an example of the reference values of the amount of change in the electrical impedance 41 that are previously determined in relation to a tissue surface between muscles.

TABLE 4

| Electrical impedance value | |
|---|---|
| change amount [kΩ] | Event name |
| 3.38 ± 0.98 | Break through fascia |

Similarly with steps S16 to S19 according to one embodiment described above, the identification means 22 calculates the amount of change in the electrical impedance 41 within a predetermined period of time, and identifies whether the type of biological tissue 9 changes, based on the reference value of the amount of change in the electrical impedance 41 shown in Table 4. The identification result is stored as a second identification result 44 in the auxiliary storage device 12, for example.

### Other Configurations

The present invention is described above based on the specific embodiments; however, the present invention is not limited to the embodiments described above.

In the above embodiments, as the type of biological tissue 9, an extraneural biological tissue (muscle or adipose tissue) and an intraneural biological tissue (nerve sheath or nerve itself) are identified, and a muscle and a biological tissue between muscles (fascia) are identified; however, the type of biological tissue 9 to be identified is not limited thereto. The present invention can be applied by using, as targets, various biological tissues whose electrical impedance can be measured. The application targets of the present invention include not only the field of anesthesiology, but also fields other than anesthesiology.

The biological tissue 9 is also not limited to living bodies, such as humans or animals, but may be biological tissue samples collected from humans or animals, or imitations created by imitating biological tissues. The present invention can also be applied to such biological tissue samples or imitations. For example, training of puncture techniques by operators or training of nerve blocks can be performed using such samples or imitations, without using living bodies. In addition, the present invention can be applied to such samples or imitations to process the samples or imitations. The step of processing a sample or imitation includes pouring a liquid between membranes.

In the above embodiments, the reference value of the amount of change in the electrical impedance 41 shown in Table 2 is used when it is determined whether the type of biological tissue 9 changes; however, the reference value of the amount of change used for determination is not limited thereto. For example, when the type of biological tissue 9 located at the tip of the electrode needle 3 changes from a first tissue to a second tissue, the measured value of the electrical impedance of the first tissue is taken as EI₁, and the measured value of the electrical impedance of the second tissue is taken as EI₂. The reference value of the amount of change shown in Table 2 is determined from |EI₂−EI₁|, which is the absolute value of the difference between the two reference values shown in Table 1. However, the reference value of the amount of change used for determination may be determined, for example, from |EI₂−EI₁|/EI₁ (or |EI₂−EI₁|/EI₂), which is a relative value. This relative value is an effective index indicating the rate at which the electrical impedance value changes in terms of the electrical impedance value of the first tissue. The same applies to the reference value of the amount of change shown in Table 4.

In the above embodiments, the internal electrode needle 31 of the electrode needle 3 has the hollow space 35; however, the internal electrode needle 31 may be solid. The step of injecting a drug solution (e.g., anesthetic 7) around the biological tissue 9 (target tissue T) through the hollow space 35 of the internal electrode needle 31 is optional.

In the above embodiments, the measured value 41 of electrical impedance is displayed in the display unit 14; however, the information displayed in the display unit 14 is not limited to the measured value 41 of electrical impedance. The display unit 14 may display, for example, the average electrical impedance of biological tissues 9, the contents of the reference value database 42, and the amount of change in the electrical impedance 41 calculated in step S16.

In the above embodiments, the identification device 2 is realized as an integrated device; however, the identification device 2 does not need to be an integrated device. The processor, memory, auxiliary storage device 12, and the like may be arranged in different places, and they may be connected with each other through a network. The input unit 13, display unit 14, and notification unit 16 also do not need to be arranged in one place; they may be arranged in different places and may be communicably connected with each other through a network.

Some or all of the functions of the data processing means 11 and the data items in the auxiliary storage device 12 may be in the cloud in an external server device (not shown) connected though the communication I/F unit 15. For example, the identification means 22 may be provided in an external server device.

In the above embodiments, the means 21 and 22 that constitute the data processing means 11 are each realized by software; however, part or the whole of these means 21 and 22 may be realized as hardware. The processing of the means 21 and 22 that constitute the data processing means 11 does not need to be processed by a single processor, but may be distributed to plural processors and processed.

In the above embodiments, a tablet terminal, a smartphone, or the like is used to constitute the identification device 2; however, a general-purpose computer, such as a personal computer, may be used to constitute the identification device 2.

### EXAMPLES

The Examples of the present invention are shown below to further clarify the features of the present invention.

### Example 1

In Example 1, the electrical impedance was measured outside and inside the sciatic nerve by advancing a bipolar electrode needle to the sciatic nerve under the guidance of ultrasound images. Then, a sciatic nerve block was performed at the position of the tip of the electrode needle using a stained local anesthetic, and it was verified whether the sciatic nerve block was properly performed.

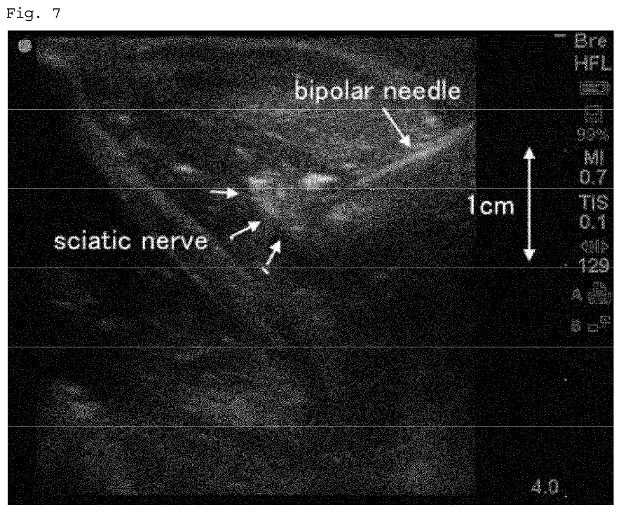

FIG. 7 shows an example of ultrasound images. In the figure, the target sciatic nerve tissue “sciatic nerve” is indicated by an arrow, and the tip of the bipolar nerve block needle “bipolar needle” is indicated by an arrow.

Method

The experiment was performed on 3 rabbits (3.06 kg) under general anesthesia. After securing the ear vein, anesthesia was introduced using propofol. After infiltration of lidocaine, tracheostomy was performed, and mechanical ventilation was managed. General anesthesia was maintained by continuous injection of propofol, and the depth of anesthesia was confirmed by eyelid reflex.

The ultrasound application area and the electrode needle puncture site were shaved, and 1% lidocaine was infiltrated into the skin to reduce pain at the puncture site of the nerve block. The electrical impedance values (EI values) were measured in tissues outside the sciatic nerve (muscle and adipose tissue) and tissues inside the sciatic nerve (sciatic nerve sheath and sciatic nerve itself). The measurement was performed while being visualized under ultrasound images using a bipolar nerve block needle (27130020, 21G×100 mm, Hakko Co., Ltd.) based on the ultrasound in-plane approach (HFL 38×13-6 MHz linear array probe, US Fujifilm Sonosite, Inc.).

Using high-frequency waves with a frequency of 1 kHz and an amplitude of 1 V, the electrical impedance was measured using an impedance analyzer (IM3570, Hioki E.E. Corporation). The frequency and potential used for this measurement are at levels that do not cause pain to the human body. Starting from the entry of the needle into the skin and continuing until the needle reached the nerve, the change in electrical impedance values every hundredth of a second was recorded on video. The electrical impedance values were measured 43 times in total in the left and right sciatic nerves of each of the 3 rabbits.

Next, based on the measured electrical impedance values, a sciatic nerve block was performed on both the left and right sides of each rabbit using a stain-containing local anesthetic (0.9 ml of 1% lidocaine and 0.1 ml of blue ink; total volume: 1 ml).

Until the electrical impedance changed, the bipolar nerve block needle was advanced to the sciatic nerve under ultrasound images. The advancement of the needle was stopped when the electrical impedance changed, and a stained local anesthetic was injected. At the end of the experiment, the rabbits were euthanized by intravenous injection of excess barbituric acid. After euthanasia, one side of the nerve block site was dissected to evaluate whether the stained local anesthetic had been correctly injected into the target area. The other side of the tissue at the sciatic nerve block site was stored at −80° C. Next, cryostat sections were prepared, and the local anesthesia position was observed with the naked eye.

Statistical Analysis

The study population was described using descriptive statistics. The statistical analysis was performed using R software version 2.10.1 (R Foundation for Statistical Computing, Austria). The Mann-Whitney U test was performed to compare central value relative electrical impedance variation between “intraneural electrical impedance” and “extraneural electrical impedance.” P-values were two-sided, and 95% confidence intervals were calculated where relevant.

Results

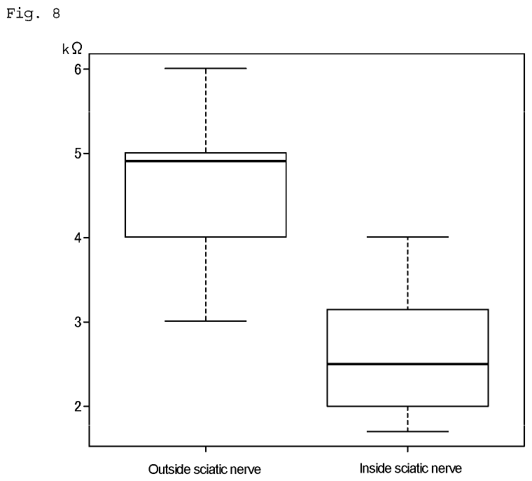

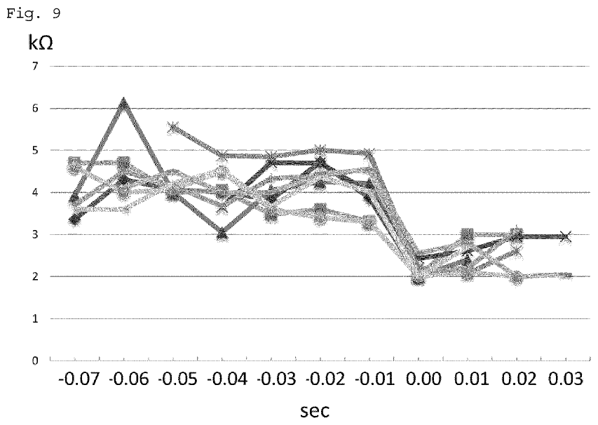

FIG. 8 is a graph showing the average of electrical impedance values inside and outside the sciatic nerve. FIG. 9 is a graph showing the time change in electrical impedance values during puncture.

As shown in FIG. 8, the mean±standard deviation of the electrical impedance values outside the sciatic nerve was 4.51±0.71 kΩ (minimum value 3 kΩ to maximum value 6 kΩ), and the mean±standard deviation of the electrical impedance values inside the sciatic nerve was 2.68±0.67 kΩ (minimum value 1.7 kΩ to maximum value 4 kΩ).

As shown in FIG. 9, even when the needle moved forward in the muscle, the electrical impedance value was stable until immediately before the needle tip entered the sciatic nerve region. Thereafter, the electrical impedance value was significantly reduced. At this point of time, stained 1% lidocaine was injected on both sides of the sciatic nerve. The presence of the stained local anesthetic injected around the sciatic nerve was visually confirmed. In frozen sections obtained from the dissected rabbits, the presence of the stained local anesthetic injected around the sciatic nerve was confirmed.

Discussion

In this Example, high-frequency waves with a frequency of 1 kHz and an amplitude of 1 V were used to measure continuous electrical impedance values. This setting is much lower than the setting used in the clinical nerve block setting. Further, a bipolar needle was used to detect the change in electrical impedance during the advancement of the needle. The use of the high-frequency waves, which allowed continuous observation of the change in electrical impedance in increments of 1 ms, made it possible to stop the advancement of the needle tip immediately before the electrode needle entered the sciatic nerve. This also made it possible to avoid significant damage to the sciatic nerve. It was confirmed by both direct observation and observation of frozen sections that the stained local anesthetic was located on the surface of the sciatic nerve.

### Example 2

In Example 2, a bipolar electrode needle was advanced in the transversus abdominis plane (TAP) under the guidance of ultrasound images, and the electrical impedance was measured for each of the internal oblique muscle and a tissue surface between the internal oblique muscle and the transverse abdominal muscle. Then, a transversus abdominis plane block (TAP block) was performed at the position of the tip of the electrode needle, and it was verified whether the TAP block was properly performed.

The transversus abdominis plane block is a peripheral nerve block for anesthesia of the abdominal wall. The TAP block is designed to anesthetize the nerves passing between the internal oblique muscle and the transverse abdominal muscle, and to block the sensation in the anterior abdominal wall (nerve range: T6 to L1). A local anesthetic is injected into the tissue surface between the internal oblique muscle and the transverse abdominal muscle to thereby anesthetize these nerves. The difficulty with the TAP block is that the tip of the puncture needle is placed on the thin tissue surface between the muscles.

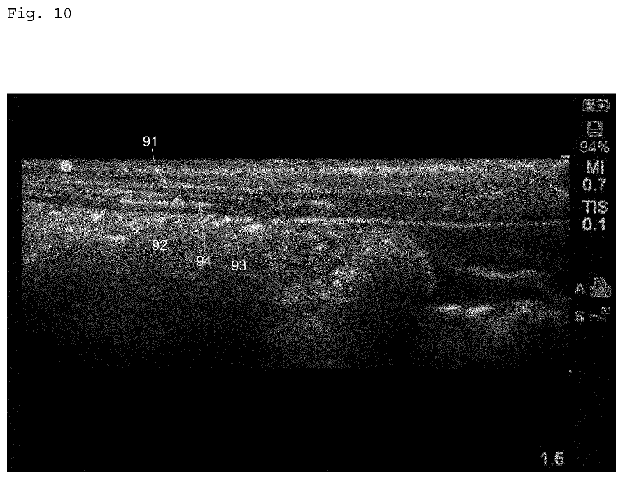

FIG. 10 shows an example of ultrasound images. In this figure, sign 91 denotes the external oblique muscle, sign 92 denotes the internal oblique muscle, and sign 93 denotes the transverse abdominal muscle. Sign 94 denotes the tissue surface.

Method

The experiment was performed in the same manner as in Example 1 described above. The electrical impedance values (EI values) were measured in a muscle (internal oblique muscle) and between two muscles (tissue surface between the internal oblique muscle and the transverse abdominal muscle). By recording the electrical impedance values on video every hundredth of a second and playing the video, the change in the electrical impedance values was recorded until the needle reached the tissue surface between the internal oblique muscle and the transverse abdominal muscle from inside the internal oblique muscle. The electrical impedance values were measured 52 times in total on the right and left sides of the abdomen of three rabbits.

Next, based on the results of the measured electrical impedance, a transversus abdominis plane block (TAP block) was performed using a stained local anesthetic. While being visualized under ultrasound images, a bipolar needle was advanced in the direction from the internal oblique muscle to the tissue surface between the internal oblique muscle and the lateral abdominal muscle, and stopped when the electrical impedance value changed. Then, a stained local anesthetic (1% lidocaine and blue ink: 1 ml in total) was injected. Thereafter, it was evaluated using ultrasound images whether the local anesthetic had been correctly injected into the target area. At the end of the experiment, the rabbits as experiment animals were euthanized by intravenous injection of excess barbituric acid. After euthanasia, the rabbits were dissected, and the injection site of the local anesthetic was observed to evaluate whether the stained local anesthetic had been correctly injected into the target area.

Statistical Analysis

The analysis was performed in the same manner as in Example 1 described above. The Mann-Whitney U test was performed to compare central value relative electrical impedance variation between “intramuscular electrical impedance” and “tissue surface electrical impedance.” P-values were two-sided, and 95% confidence intervals were calculated where relevant.

It was determined that the experimental results of each electrical impedance value were not normally distributed, and the nonparametric method was used in the analysis. First, the Kruskal-Wallis test was used to determine whether the electrical impedance value at T0 was different from the others. Next, post-hoc multiple comparisons (Steel-Dwass nonparametric method) were performed, the point where the electrical impedance value changed was set to 0 seconds, and it was determined whether the electrical impedance value was different from other points every 0.01 seconds until 0.05 seconds ago.

Results

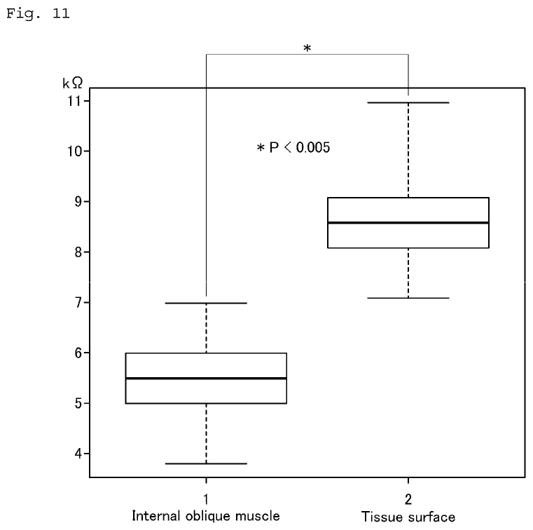

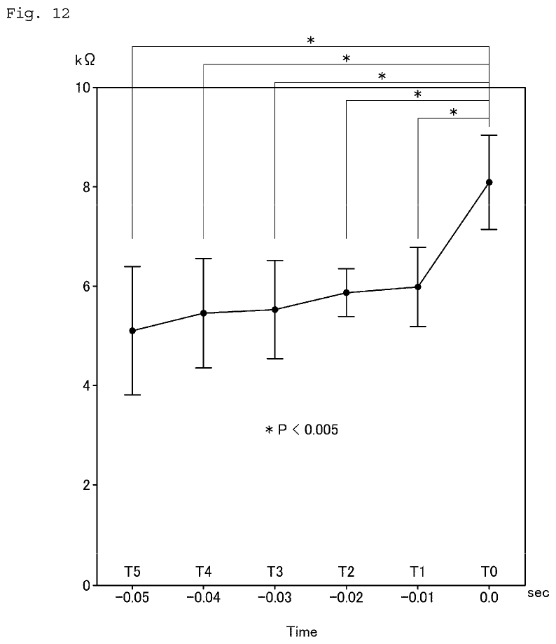

FIG. 11 is a graph showing the average of electrical impedance values in the internal oblique muscle and the tissue surface. FIG. 12 is a graph showing the time change in electrical impedance values during puncture.

As shown in FIG. 11, the mean±standard deviation of the electrical impedance values of the internal oblique muscle was 5.41±0.66 kΩ (minimum value 3.8 kΩ to maximum value 7 kΩ), and the mean±standard deviation of the electrical impedance values of the tissue surface between the internal oblique muscle and the transverse abdominal muscle was 8.79±0.94 kΩ (minimum value 7.1 kΩ to maximum value 11 kΩ). There was a significant difference between these two populations (p<0.001).

As shown in FIG. 12, even when the needle moved forward in the muscle, the electrical impedance of the muscle was stable until the needle tip entered the tissue surface. Thereafter, the electrical impedance value was significantly increased (p<0.005). As a result of injecting stained 1% lidocaine between the internal oblique muscle and the transverse abdominal muscle at this point of time, the local anesthetic was visually confirmed in the ultrasound image. After the rabbits were dissected, the presence of the stained local anesthetic injected between the internal oblique muscle and the transverse abdominal muscle was visually confirmed.

Discussion

In this Example, it was confirmed that the electrical impedance value of the internal oblique muscle was stable. It was also confirmed that the electrical impedance changed to the tissue surface in the tissue surface between the internal oblique muscle and the lateral abdominal muscle. This difference was sufficient to detect the position of the needle tip. As a result of actually injecting a local anesthetic at this point of time, it could be confirmed by ultrasound images and visual observation that the local anesthetic was injected between the muscles. The transversus abdominis plane (TAP) block is a peripheral nerve block designed to anesthetize sensory nerves, and this provides the anterior abdominal wall lying between the internal oblique muscle and the transverse abdominal muscle. The fact that the local anesthetic could be accurately injected between the muscles suggested that TAP blocks can be performed sufficiently by measurement of electrical impedance values.

### Example 3

When the type of biological tissue located at the tip of the electrode needle changes from a first tissue to a second tissue, the measured value of the electrical impedance of the first tissue is taken as EI₁, and the measured value of the electrical impedance of the second tissue is taken as EI₂, whether the type of biological tissue changes can be determined based on the amount of change in electrical impedance |EI₂−EI₁|. The amount of change in electrical impedance used for the determination can also be based on a relative value |EI₂−EI₁|/EI₁ (or |EI₂−EI₁|/EI₂) in place of the absolute value |EI₂−EI₁|. In Example 3, a bipolar electrode needle was advanced to the sciatic nerve under guidance of ultrasound images by the same procedure as in Example 1, thereby verifying the change in the measured value of electrical impedance when the position of the tip of the electrode needle changed from outside the sciatic nerve to inside the sciatic nerve.

Method

The experiment was performed in the same manner as in Example 1 described above. The experiment was performed on each of 5 rabbits. For all of the 5 rabbits, the measured values of electrical impedance were obtained when the position of the tip of the electrode needle changed from the muscle, which is a tissue outside the sciatic nerve, to the sciatic nerve sheath, which is a tissue inside the sciatic nerve.

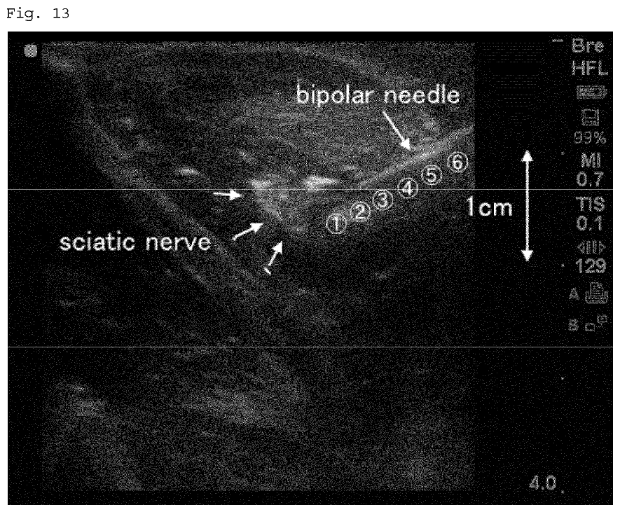

FIG. 13 shows an example of ultrasound images. In this figure, the target sciatic nerve tissue “sciatic nerve” is indicated by an arrow, and the bipolar nerve block needle “bipolar needle” is indicated by an arrow. The tip of the bipolar nerve block needle was advanced towards the sciatic nerve along from a position indicated by circled number 6 to a position indicated by circled number 1 in the figure. As a result, the electrical impedance values were measured when the tip of the bipolar nerve block needle was located at each of the 6 positions indicated by the circled numbers.

Thereafter, by the same procedure as in Example 1, a sciatic nerve block was performed at the position of the tip of the electrode needle using a stained local anesthetic, and it was confirmed that the sciatic nerve block was properly performed. At the end of the experiment, the rabbits were euthanized, cryostat sections were prepared, and the local anesthesia position was observed with the naked eye. As a result, it was confirmed that the position indicated by circled number 1 corresponded to the sciatic nerve sheath, and that the position indicated by circled number 2 corresponded to the muscle outside the sciatic nerve.

In the following description regarding Example 3, based on time (T₀) when the tip of the electrode needle is located in the sciatic nerve sheath, 5 times before this reference time T₀ are represented by T₁ to T₅. For example, the time at the position of the tip of the electrode needle indicated by circled number 6 is represented by T₅, and the measured value of electrical impedance at that time is represented by EI@T₅. Similarly, the time at the position of the tip of the electrode needle indicated by circled number 2 is represented by T₁, and the measured value of electrical impedance at that time is represented by EI@T₁. The time at the position of the tip of the electrode needle indicated by circled number 1 is represented by T₀, and the measured value of electrical impedance at that time is represented by EI@T₀. 5 times T₁ to T₅ were each in increments of 0.01 seconds, and the time difference between time T₀ and time T₁ was 0.01 seconds.

Results

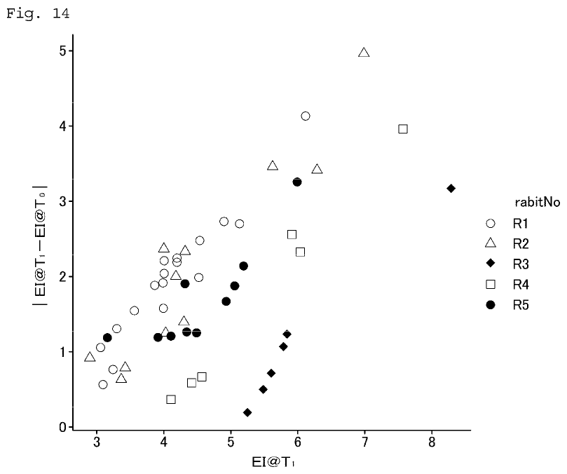

FIG. 14 is a graph showing the change in the measured value of electrical impedance when the position of the tip of the electrode needle changes from the muscle to the sciatic nerve sheath. The horizontal axis of the graph is the measured value of electrical impedance (unit: kΩ) when the tip of the electrode needle is located in the muscle outside the sciatic nerve immediately before entering the sciatic nerve. The measured value of electrical impedance at this time is represented by EI@T₁. The vertical axis of the graph is the amount of change in the measured value of electrical impedance (unit: kΩ) when the position of the tip of the electrode needle changes from the muscle outside the sciatic nerve immediately before entering the sciatic nerve to the sciatic nerve sheath. The amount of change in the measured value of electrical impedance is represented by |EI@T₁−EI@T₀|.

The graph of FIG. 14, which shows the change in the measured value of electrical impedance, indicated the following two matters:
When the measured value of the electrical impedance of the muscle EI@T₁ is high, the amount of change in the measured value of electrical impedance |EI@T₁−EI@T₀| is also large.
There is a positive correlation (correlation coefficient r=0.63) between the measured value of the electrical impedance of the muscle EI@T₁ and the amount of change in the measured value of electrical impedance |EI@T₁−EI@T₀|.

Discussion

The graph of FIG. 14 showed that there was a positive correlation between the value shown on the horizontal axis and the value shown on the vertical axis of the graph. This confirmed that in this Example, when the position of the tip of the electrode needle changed from the muscle to the sciatic nerve sheath, the amount of change in the measured value of electrical impedance |EI@T₁−EI@T₀| tended to increase as the measured value of the electrical impedance of the muscle EI@T₁ increased.

The paracentesis assistance system according to one embodiment of the present invention determines whether the type of biological tissue changes. For this determination, the amount of change in electrical impedance within a predetermined period of time is used. In variations of the present invention, the amount of change in electrical impedance used for determination is calculated based on the relative value |EI₂−EI₁|/EI₁ or |EI₂−EI₁|/EI₂. This relative value is an effective index indicating the rate at which the electrical impedance value changes in terms of the electrical impedance value of the first tissue A.

Regarding the graph of FIG. 14, since there is a positive correlation between the value shown on the vertical axis and the value shown on the horizontal axis of the graph, the ratio of these values is taken. The ratio is represented by |EI@T₁−EI@T₀|/EI@T₁, and indicates the rate at which the electrical impedance value changes in terms of the electrical impedance value of the muscle when the position of the tip of the electrode needle changes from the muscle (time T₁) to the sciatic nerve sheath (time T₀). Therefore, it was supported by the graph of FIG. 14 that the ratio of the value shown on the vertical axis to the value shown on the horizontal axis of the graph, |EI@T₁−EI@T₀|/EI@T₁ (or |EI@T₁−EI@T₀|/EI@T₀), was effective as an index used for the determination of the amount of change in electrical impedance. Due to the use of the ratio |EI@T₁−EI@T₀|/EI@T₁ (or |EI@T₁−EI@T₀|/EI@T₀) for the determination of the amount of change in electrical impedance, the steep change in electrical impedance values that occurs between different tissues can be identified.

### REFERENCE SIGNS LIST

1. Measurement device
2. Identification device
3. Electrode needle
4. Ultrasound probe
5. Ultrasound diagnostic device
6. Electrical stimulus generator
7. Anesthetic
9. Biological tissue
10. Paracentesis assistance system
11. Data processing means
12. Auxiliary storage device
13. Input unit
14. Display unit
15. Interface (I/F) unit
16. Notification unit
21. Measurement operation control means
22. Identification means
31. Electrode (internal electrode needle)
31A. End part of internal electrode needle
32. Electrode (external electrode needle)
32A. End face of external electrode needle
33, 34. Insulation layer
35. Hollow space
37, 38. Connection code
41. Electrical impedance
42. Reference value database
43, 44. Identification result
51. Ultrasound image
71. Drug solution tube
91. External oblique muscle
92. Internal oblique muscle
93. Transverse abdominal muscle
94. Tissue surface
P. Paracentesis assistance program
T. Target tissue

1. A paracentesis assistance system comprising:
a measurement device that applies high-frequency waves to at least two electrodes of an electrode needle inserted into a biological tissue, and repeatedly measures the electrical impedance of the biological tissue where the electrodes are located, the electrodes being arranged at the tip of the electrode needle in a longitudinal direction; and
an identification device that identifies the type of biological tissue based on the temporal change in the repeatedly measured electrical impedance.

2. The paracentesis assistance system according to claim 1, wherein the identification device calculates a time average of the electrical impedance within a predetermined period of time, and compares the calculated time average with a database that associates the type of biological tissue with a reference value of the time average, thereby identifying the type of biological tissue.

3. The paracentesis assistance system according to claim 2, further comprising a notification unit that gives notification depending on an identification result by the identification device.

4. The paracentesis assistance system according to claim 3, wherein the notification unit gives notification when the calculated time average is outside a predetermined value range including the reference value.

5. The paracentesis assistance system according to any one of claims 1 to 4, wherein the identification device calculates the amount of change in the electrical impedance within a predetermined period of time, and identifies that the type of biological tissue changes when the calculated amount of change is within a predetermined value range.

6. The paracentesis assistance system according to claim 5, wherein the identification device calculates the amount of change in the electrical impedance within a predetermined period of time based on |EI₂−EI₁|/EI₁ or |EI₂−EI₁|/EI₂, wherein EI₁ is the measured value of the electrical impedance of a first tissue, and EI₂ is the measured value of the electrical impedance of a second tissue, when the type of biological tissue located at the tip of the electrode needle changes from the first tissue to the second tissue.

7. The paracentesis assistance system according to claim 5 or 6, wherein the identification device identifies whether the electrodes are located in a nerve tissue.

8. The paracentesis assistance system according to claim 5 or 6, wherein the identification device identifies whether the electrodes are located in a biological tissue between muscles.

9. The paracentesis assistance system according to claim 7, further comprising an electrical stimulus generator that applies an electrical pulse to the electrodes to stimulate the biological tissue.

10. The paracentesis assistance system according to claim 7, wherein the electrode needle is hollow, and an anesthetic is injected through the electrode needle into the biological tissue where the electrodes are located.

11. A paracentesis assistance method comprising:
applying high-frequency waves to at least two electrodes of an electrode needle inserted into a biological tissue, and repeatedly measuring the electrical impedance of the biological tissue where the electrodes are located, the electrodes being arranged at the tip of the electrode needle in a longitudinal direction; and
identifying the type of biological tissue based on the temporal change in the repeatedly measured electrical impedance.

12. A program for causing a computer to realize:
a function of applying high-frequency waves to at least two electrodes of an electrode needle inserted into a biological tissue, and repeatedly measuring the electrical impedance of the biological tissue where the electrodes are located, the electrodes being arranged at the tip of the electrode needle in a longitudinal direction; and
a function of identifying the type of biological tissue based on the temporal change in the repeatedly measured electrical impedance.
